(19) United States
(12) Patent Application Publication
(10) Pub. No.: US 2026/0325049 A1
(43) Pub. Date: Oct. 22, 2026

(54) IDENTIFYING CEREBROVASCULAR DISEASE AND MEDIAL TEMPORAL ATROPHY

(71) Applicant: QUANTALX NEUROSCIENCE LTD, Kfar Sava (IL)

(72) Inventors: Hilla FOGEL, Mazor (IL); Iftach DOLEV, Zur Moshe (IL); Ofri LEVY-LAMDAN, Ramat Gan (IL); Noa ZIFMAN, Kfar Sava (IL)

(21) Appl. No.: 19/702,072

(22) Filed: Jun. 9, 2026

Related U.S. Application Data
(63) Continuation of application No. PCT/IB2024/062443, filed on Dec. 10, 2024.
(60) Provisional application No. 63/608,422, filed on Dec. 11, 2023.

(51) Int. Cl. A61B 5/377; A61B 5/00; A61B 5/372

(52) U.S. Cl. CPC A61B 5/377; A61B 5/372; A61B 5/4088; A61B 5/4842

(57) ABSTRACT

Apparatus and methods are described including a system that includes multiple electrodes configured to record respective signals produced, by a brain of a patient, in response to magnetic stimulation of the brain. A processor is configured to receive the signals, and, by processing the signals, ascertain that the patient has cerebrovascular disease. The processor generates an output indicating that the patient has cerebrovascular disease. Other applications are also described.

### CROSS-REFERENCE TO RELATED APPLICATIONS

The present application is a continuation of PCT Application PCT/IB2024/062443 to Fogel et al., filed Dec. 10, 2024 (published as WO 25/126038), entitled “Identifying cerebrovascular disease and medial temporal atrophy,” which claims priority to US Provisional Application 63/608,422 to Fogel et al., filed Dec. 11, 2023, entitled “Identifying cerebrovascular disease and medial temporal atrophy,” which is incorporated herein by reference.

### FIELD

The present invention relates to the field of medical diagnostics, particularly with respect to conditions associated with cognitive impairment (e.g., dementia).

### BACKGROUND

The use of electrophysiological measurements to characterize and monitor brain network activity has been used extensively over the last seven decades. Electrophysiological measurements can be generally divided into two groups of parameters: network integrity, i.e., the connectivity and coherence of the network, and network plasticity, also referred to as “neuroplasticity” or “brain plasticity.” Network connectivity depends on the synchronous activation of neurons. Network coherence refers to the level of synchrony between two or more brain regions and is used to determine the strength of connectivity between specific brain regions. Neuroplasticity is an ability of the brain to continuously adapt its functional and structural organization to changing requirements. Neuronal plasticity allows the brain to reorganize neuronal networks in response to environmental stimulation, to remember information and to recover from brain and spinal cord injuries. Neuronal plasticity is essential to the establishment and maintenance of brain circuitry.

Magnetic simulation is a non-invasive brain stimulation method that allows the study of human cortical function in vivo. Using magnetic stimulation for examining human cortical functionality is enhanced by combining such stimulation with registration of an electrical evoked response, such as an electroencephalograph (EEG). EEG provides an opportunity to directly measure the cerebral response to magnetic stimulation, measuring the cortical evoked potential. An important feature of the evoked potential topography is that even though only one cortical hemisphere is stimulated, bi-hemispheric EEG responses are evoked with different features. Magnetic-stimulation-evoked activity propagates from the stimulation site ipsilaterally via association fibers, contralaterally via transcallosal fibers, and to subcortical structures via projection fibers. A single stimulating pulse (delivered over the primary motor cortex (M1), for example) results in a sequence of positive and negative EEG peaks at specific latencies (typically, negative peaks at 45 ms (N45) and 100 ms (N100) after stimulation, and positive peaks at 60 ms (P60) and 180 ms (P180) after stimulation). This pattern of response indicates synaptic activity. These evoked cortical potentials last for up to 300 ms both in the vicinity of the stimulation and in remote interconnected brain areas.

Cerebrovascular disease (e.g., cerebral small vessel disease (CSVD)), which is associated with cognitive impairment (e.g., dementia), has various indications that can be detected using medical imaging. Such indications include leukoaraiosis, which is a change in appearance of white matter near the lateral ventricles such that the white matter appears hyperintense or hypointense in medical images. Such indications further include cerebral microbleeds and lacunar infarction. (A lacunar infarct is a small (e.g., 2-15 mm diameter) noncortical infarct caused by the occlusion of a penetrating branch of a large cerebral artery within the brain.) Medial temporal atrophy (MTA), also known as medial temporal lobe atrophy, refers to a loss of mass in the hippocampal area of the brain, and is also associated with cognitive impairment.

### SUMMARY

For a patient with suspected or confirmed cognitive impairment, it is typically important to identify or rule out cerebrovascular disease and/or medial temporal atrophy. However, conventional techniques for doing this, such as computed tomography (CT) imaging and magnetic resonance imaging (MRI), have limited accuracy and/or limited practicality (e.g., due to high costs or inaccessibility).

To address this challenge, embodiments of the present invention provide a system including multiple electrodes and a processor. The electrodes are configured to record respective signals produced, by a brain of a patient, in response to magnetic stimulation of the brain, such as the primary motor cortex or dorsolateral prefrontal cortex of the brain. The processor is configured to receive the signals and, by processing the signals, ascertain that the patient has cerebrovascular disease and/or ascertain that the patient has medial temporal atrophy. The processor is further configured to output an output indicating that the patient has cerebrovascular disease and/or that the patient has medial temporal atrophy.

There is therefore provided, in accordance with some embodiments of the present invention, a system including multiple electrodes configured to record respective signals produced, by a brain of a patient, in response to magnetic stimulation of the brain. The system further includes a processor configured to receive the signals, to ascertain that the patient has cerebrovascular disease by processing the signals, and to output an output indicating that the patient has cerebrovascular disease.

In some embodiments, the magnetic stimulation includes magnetic stimulation of a primary motor cortex of the brain.

In some embodiments, the magnetic stimulation includes magnetic stimulation of a dorsolateral prefrontal cortex of the brain.

In some embodiments, the processor is further configured to identify a severity of the cerebrovascular disease by processing the signals, and the output indicates the severity.

In some embodiments, the processor is configured to process the signals by:
computing one or more measures of neurophysiological activity exhibited in the signals, and
ascertaining that the patient has cerebrovascular disease based on the measures.

In some embodiments, the measures of neurophysiological activity include a waveform adherence measure, which is based on a similarity between a waveform of a portion of the signals and a waveform of a corresponding portion of a benchmark signal.

In some embodiments, the measures of neurophysiological activity include a cortical excitability measure, which is based on an amplitude of the signals.

In some embodiments, the processor is further configured to identify subset indications of multiple indications of cerebrovascular disease by processing the signals, and the output indicates that the patient has at least one of the subset indications of the multiple indications.

In some embodiments, the subset indications of the multiple indications include leukoaraiosis, cerebral microbleeds, and lacunar infarction.

There is further provided, in accordance with some embodiments of the present invention, a method including receiving multiple signals, which were produced by a brain of a patient in response to magnetic stimulation of the brain and were recorded by respective electrodes. The method further includes, by processing the signals, ascertaining that the patient has cerebrovascular disease, and outputting an output indicating that the patient has cerebrovascular disease.

There is further provided, in accordance with some embodiments of the present invention, a computer software product including a tangible non-transitory computer-readable medium in which program instructions are stored. The instructions, when read by a processor, cause the processor to receive multiple signals, which were produced by a brain of a patient in response to magnetic stimulation of the brain and were recorded by respective electrodes. The instructions further cause the processor to ascertain that the patient has cerebrovascular disease by processing the signals, and to output an output indicating that the patient has cerebrovascular disease.

There is further provided, in accordance with some embodiments of the present invention, a system including multiple electrodes configured to record respective signals produced, by a brain of a patient, in response to magnetic stimulation of the brain. The system further includes a processor configured to receive the signals, to ascertain that the patient has medial temporal atrophy by processing the signals, and to output an output indicating that the patient has medial temporal atrophy.

In some embodiments, the magnetic stimulation includes magnetic stimulation of a primary motor cortex of the brain.

In some embodiments, the magnetic stimulation includes magnetic stimulation of a dorsolateral prefrontal cortex of the brain.

In some embodiments, the processor is configured to process the signals by:
computing one or more measures of neurophysiological activity exhibited in the signals, and
ascertaining that the patient has medial temporal atrophy based on the measures.

In some embodiments, the measures of neurophysiological activity include a cortical excitability measure, which is based on an amplitude of a portion of the signals.

In some embodiments, the processor is further configured to identify a severity of the medial temporal atrophy by processing the signals, and the output indicates the severity.

In some embodiments, the processor is configured to identify the severity based on an input indicating whether the patient is cognitively impaired.

There is further provided, in accordance with some embodiments of the present invention, a method including receiving multiple signals, which were produced by a brain of a patient in response to magnetic stimulation of the brain and were recorded by respective electrodes. The method further includes, by processing the signals, ascertaining that the patient has medial temporal atrophy, and outputting an output indicating that the patient has medial temporal atrophy.

There is further provided, in accordance with some embodiments of the present invention, a computer software product including a tangible non-transitory computer-readable medium in which program instructions are stored. The instructions, when read by a processor, cause the processor to receive multiple signals, which were produced by a brain of a patient in response to magnetic stimulation of the brain and were recorded by respective electrodes, to ascertain that the patient has medial temporal atrophy by processing the signals, and to output an output indicating that the patient has medial temporal atrophy.

The present invention will be more fully understood from the following detailed description of embodiments thereof, taken together with the drawings, in which:

### BRIEF DESCRIPTION OF THE DRAWINGS

FIG. 1 is a schematic illustration of a clinician performing a diagnostic procedure on a patient using a diagnostic system, in accordance with some embodiments of the present invention;

FIG. 2 is a flow diagram for a method for ascertaining whether a patient has cerebrovascular disease or medial temporal atrophy, in accordance with some embodiments of the present invention;

FIG. 3 is a flow diagram for a measure-computing step, in accordance with some embodiments of the present invention;

FIGS. 4A, 4B, 4C, 4D, 4E, 4F, 4G, and 4H show experimental results demonstrating the utility of embodiments of the present invention; and

FIGS. 5A, 5B, and 5C show experimental results demonstrating the utility of embodiments of the present invention.

### DETAILED DESCRIPTION

### Overview

For a patient with suspected or confirmed cognitive impairment, it is typically important to identify or rule out cerebrovascular disease and/or medial temporal atrophy. However, conventional techniques for doing this, such as computed tomography (CT) imaging and magnetic resonance imaging (MRI), have limited accuracy and/or limited practicality (e.g., due to high costs or inaccessibility).

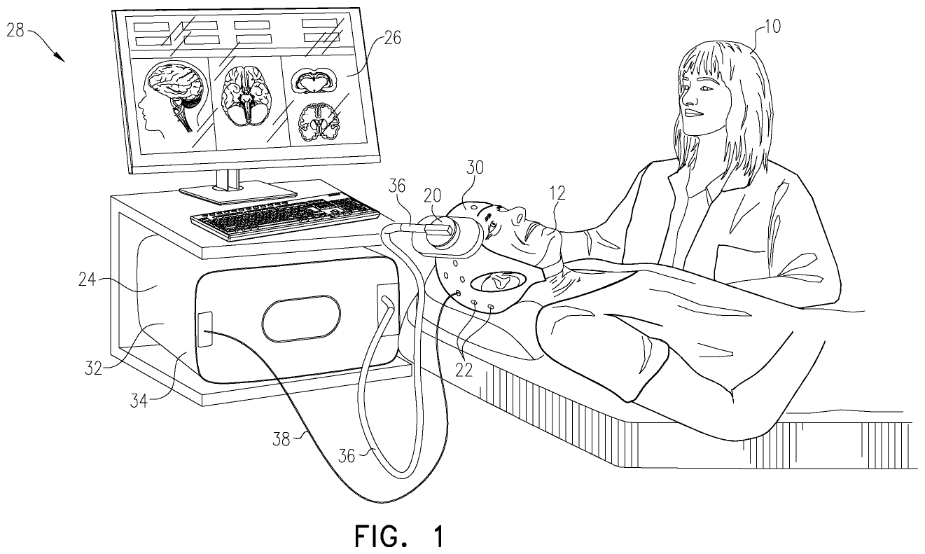

To address this challenge, embodiments of the present invention analyze the neurophysiological response of the patient to magnetic stimulation, which, as the present inventors have proved via experimentation, is often indicative of whether the patient has cerebrovascular disease or medial temporal atrophy. In particular, embodiments of the present invention provide a diagnostic system 28, shown in FIG. 1. The system comprises a magnetic stimulation device 20, multiple electrodes 22, and a computer processor 32. The magnetic stimulation device is configured to stimulate one or more cortexes of the patient's brain. Following each stimulation, the electrodes record the resulting evoked-potential signals produced by the brain in response to the stimulation. The processor processes the signals, typically by computing one or more measures of neurophysiological activity exhibited in the signals, so as to ascertain whether the patient has cerebrovascular disease and/or whether the patient has medial temporal atrophy.

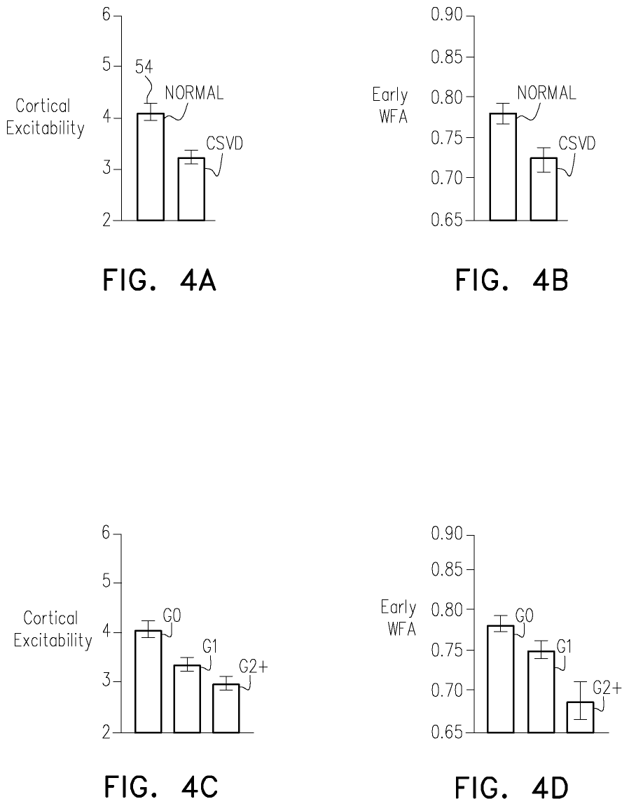

In some embodiments, to compute a measure of neurophysiological activity, the processor computes a property (e.g., a waveform or integral) of multiple evoked-potential signals as the property of a representative signal that is based on the multiple signals, such as an average of the multiple signals. Alternatively or additionally, the processor computes a statistic (e.g., an average) of the property over the signals, i.e., the processor computes the property separately for each of the signals, and then computes the statistic. Alternatively or additionally, as shown in FIG. 3, which is described below, the processor computes a statistic of the property over multiple representative signals, each of which is based on a different respective subset of the evoked-potential signals. In the context of the present description and claims, each reference to a property of multiple signals (or a property of a portion of multiple signals) encompasses all these possibilities within its scope.

### System Description

Reference is initially made to FIG. 1, which is a schematic illustration of a clinician 10 performing a diagnostic procedure on a patient 12 using a diagnostic system 28, in accordance with some embodiments of the present invention. The diagnostic procedure may be performed, for example, if patient 12 has suspected or confirmed cognitive impairment and/or is at risk for dementia (e.g., due to age or other cardiovascular risk factors). As further described below, the diagnostic procedure may identify that the patient has cerebrovascular disease and/or medial temporal atrophy.

System 28 comprises a magnetic stimulation device 20 configured for placement near the head of patient 12, e.g., by virtue of being placed over a cap 30 worn over the head. Device 20 comprises one or more coils configured to generate a magnetic field, which stimulates activity within the brain of patient 12.

In general, device 20 may be placed over any portion of the brain associated with symptoms of cerebrovascular disease, so as to stimulate that portion of the brain. Example portions include the frontal cortex (e.g., the primary motor cortex, any other portion of the motor cortex, or the dorsolateral prefrontal cortex), the sensory cortex (e.g., the visual cortex), the parietal cortex (e.g., the posterior parietal cortex), the temporal cortex (e.g., the lateral temporal cortex), and any portion of the brain connected to any of the above. Alternatively or additionally, device 20 may be placed over any portion of the brain associated with symptoms of medial temporal atrophy, such as any of the example portions listed above or the precuneus. In particular, the present inventors have found that for diagnosing cerebrovascular disease or medial temporal atrophy, it may be effective to stimulate the left or right primary motor cortex, and/or the left or right dorsolateral prefrontal cortex, of the brain. Hence, device 20 may be placed over the left or right primary motor cortex, and/or the left or right dorsolateral prefrontal cortex, of the patient's brain.

System 28 further comprises multiple electrodes 22 configured to record respective signals produced by the brain in response to the stimulation. Electrodes 22 may be coupled to the patient's head via a low-impedance adhesive material. Alternatively, electrodes 22 may be coupled to cap 30 such that, when cap 30 is fittingly placed over the patient's head, the electrodes contact the head (optionally via an impedance-reducing gel). Collectively, electrodes 22 may be referred to as an electroencephalograph (EEG) detector.

System 28 further comprises a control unit 24 comprising a signal generator 34 and other circuitry, such as analog-to-digital (A/D) conversion circuitry and/or denoising circuitry. Typically, control unit 24 is connected to stimulation device 20 via a cable 36. A computer processor 32—which may belong to control unit 24 or to an external device, such as a laptop, in communication with control unit 24 (e.g., via a universal serial bus (USB) cable)—is configured to drive signal generator 34 to generate electrical signals. These signals flow through device 20 via cable 36, thus causing the device to generate a magnetic field, which in turn evokes signals (or “potentials”) in the patient's brain. These signals are recorded by electrodes 22 and passed from the electrodes, via respective leads 38 or via wireless transfer, to the control unit. (For ease of illustration, only one lead 38 is shown in FIG. 1.) After optional denoising and digitization within control unit 24, processor 32 receives the signals.

Processor 32 is further configured to process the signals so as to ascertain whether the patient has cerebrovascular disease and/or whether the patient has medial temporal atrophy, each of which is associated with cognitive impairment. The processor additionally outputs an output indicating the diagnosis; for example, the processor may display the output on a display 26. In some embodiments, the output includes a likelihood or confidence measure associated with the diagnosis. For example, the output may specify a 90% likelihood that the patient has cerebrovascular disease and/or an 85% likelihood that the patient does not have medial temporal atrophy.

Typically, in processing the signals, the processor computes one or more measures of neurophysiological activity exhibited in the signals and ascertains whether the patient has cerebrovascular disease, and/or whether the patient has medial temporal atrophy, based on the measures. For example, the processor may compare the measures to respective thresholds or input the measures to a model, such as a neural network or logistic regression model, which is calibrated to output a diagnosis.

In some embodiments, the computed measures of neurophysiological activity include a waveform adherence measure, which quantifies the similarity between the waveform of a portion of the signals and the waveform of a corresponding portion of a benchmark signal, which may be obtained, for example, from relevant literature. Typically, the benchmark signal represents the response of a normal subject.

One such waveform adherence measure is a wide waveform adherence measure, which quantifies the similarity over a relatively long (or “wide”) portion of the signals. For example, the portion may begin 15-55 ms from the start of the signals (i.e., from the end of the stimulation) and have a duration of 300-350 ms.

Another such waveform adherence measure is a late waveform adherence measure, which quantifies the similarity over a relatively late portion of the signals. For example, the portion may begin at least 60 ms (e.g., at least 80 ms) from the start of the signals. The duration of the portion may be, for example, between 120 and 160 ms.

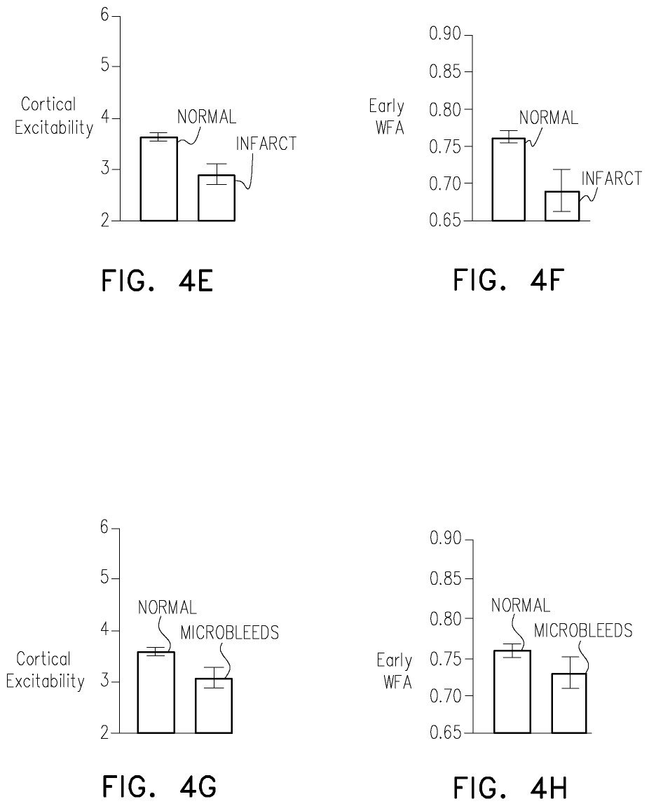

Yet another such waveform adherence measure is an early waveform adherence measure, which quantifies the similarity over a relatively early portion of the signals. For example, the portion may begin less than 50 ms from the start of the signals. The duration of the portion may be, for example, between 120 and 160 ms. As a specific example, the portion may begin 35 ms post-stimulus and have a duration of 145 ms. As described below with reference to FIGS. 4B, 4D, 4F, and 4H, experimental results have shown that a diagnosis of cerebrovascular disease or of medial temporal atrophy may be based on the early waveform adherence measure.

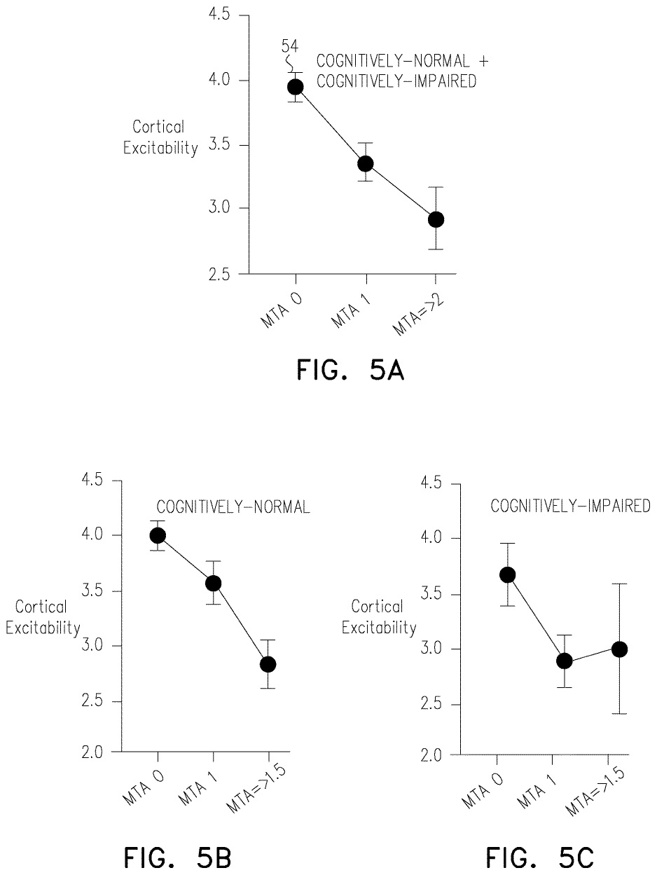

Alternatively or additionally, the computed measures of neurophysiological activity include a cortical excitability measure, which is based on an amplitude of a portion of the signals. For example, the cortical excitability measure may be based on an integral of the signals (which depends on the amplitude) or on a statistic of the amplitude, such as the mean average deviation of the amplitude. As described below with reference to FIGS. 4A, 4C, 4E, 4G, and 5A-5C, experimental results have shown that a diagnosis of cerebrovascular disease or of medial temporal atrophy may be based on the cortical excitability measure.

Alternatively or additionally, the computed measures of neurophysiological activity include a waveform excitability measure for a portion of signals, which is based both on the amplitude of the portion of the signals and on the similarity of the waveform to a benchmark waveform. Alternatively or additionally, the computed measures of neurophysiological activity include an interhemispheric connectivity measure, which quantifies the similarity between the response of the right side of the patient's brain to the stimulation and the response of the left side of the patient's brain to the stimulation, and/or a main-peak latency measure, which quantifies the latencies of one or more main peaks in the signals.

As noted above in the Background, cerebrovascular disease has various indications that can be detected using medical imaging, including leukoaraiosis, cerebral microbleeds, and lacunar infarction. In some embodiments, the processor is configured to identify a subset of these multiple indications by processing the signals, and the output indicates that the patient has at least one indication of the subset. For example, the output may indicate that the patient has leukoaraiosis, cerebral microbleeds, and/or lacunar infarction. In some embodiments, this diagnosis is based on the cortical excitability and/or early waveform adherence measures, as shown in FIGS. 4C-4H, which are described below.

In some embodiments, in addition to ascertaining that the patient has cerebrovascular disease, the processor identifies the severity of the cerebrovascular disease by processing the signals. For example, as demonstrated in FIGS. 4C-4D, which are described below, the processor may identify the severity of leukoaraiosis based on the cortical excitability and/or early waveform adherence measures.

Similarly, in some embodiments, for a diagnosis of medial temporal atrophy, the processor identifies the severity of the medial temporal atrophy, and the output indicates the severity. For example, as demonstrated in FIGS. 5A-5C, which are described below, the processor may identify the severity based on the cortical excitability measure, and/or based on an input indicating whether the patient is cognitively impaired.

In general, processor 32 may be embodied as a single processor, or as a cooperatively networked or clustered set of processors. The functionality of processor 32 may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, processor 32 may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

### Processing the Evoked Potentials

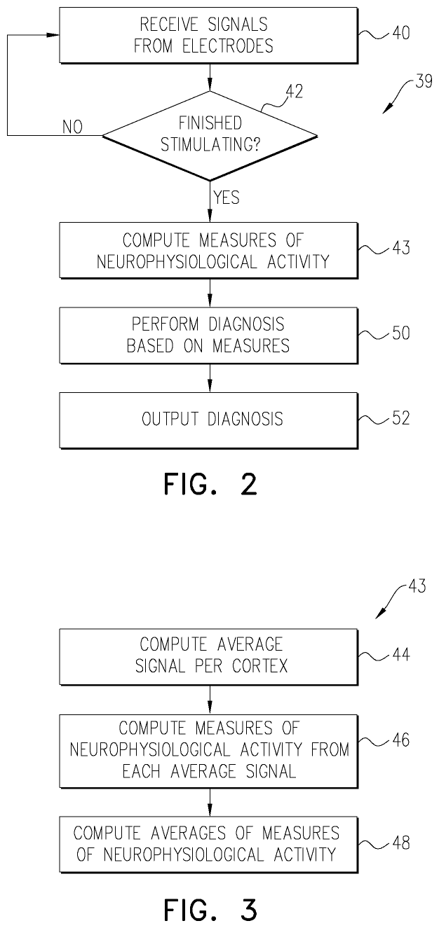

Reference is now made to FIG. 2, which is a flow diagram for a method 39 for ascertaining whether patient 12 (FIG. 1) has cerebrovascular disease or medial temporal atrophy, in accordance with some embodiments of the present invention.

Method 39 begins at a signal-receiving step 40, at which processor 32 receives respective signals recorded by electrodes 22 (FIG. 1). As described above with reference to FIG. 1, these signals were produced, by the brain of the patient, in response to magnetic stimulation of the brain.

Subsequently to receiving the signals, the processor checks, at a checking step 42, whether more stimulations are to be performed. For example, the processor may perform checking step 42 by processing an input from the clinician. If more stimulations are to be performed, the processor returns to signal-receiving step 40, and receives the evoked potentials from the next stimulation.

In general, any cortex of the patient's brain may be stimulated any number of times, and the processor may perform signal-receiving step 40 after each of these stimulations. For example, the clinician may perform several (e.g., 8-12) stimulations of the right primary motor cortex, several (e.g., 8-12) stimulations of the left primary motor cortex, several (e.g., 8-12) stimulations of the right dorsolateral prefrontal cortex, and several (e.g., 8-12) stimulations of the left dorsolateral prefrontal cortex. Typically, the stimulation protocol-including the number of repetitions, and the intensity and frequency of the stimulation signal at each stimulation site-is predetermined. Alternatively, the clinician sets the simulation protocol.

Upon ascertaining that no more stimulations are to be performed, the processor performs a measure-computing step 43, at which the processor computes one or more measures of neurophysiological activity exhibited in the signals. Subsequently, at a diagnosing step 50, the processor performs a diagnosis based on the computed measures, e.g., by comparing the measures to respective threshold measures and/or inputting the measures to a model. Finally, at an outputting step 52, the processor outputs the diagnosis.

For example, in response to the cortical excitability measure being less than a threshold, the output may indicate that the patient likely has medial temporal atrophy and, optionally, the estimated severity of the medial temporal atrophy. Conversely, in response to the cortical excitability being greater than the threshold, the output may indicate that the patient likely does not have medial temporal atrophy. Alternatively or additionally, in response to the cortical excitability and/or early waveform adherence measures being less than a threshold or less than respective thresholds, the output may indicate that the patient likely has cerebrovascular disease, with an optional additional diagnosis of leukoaraiosis, cerebral microbleeds, and/or lacunar infarction. Conversely, in response to one or both of these measures being greater than the threshold(s), the output may indicate that the patient likely does not have cerebrovascular disease.

Reference is now made to FIG. 3, which is a flow diagram for measure-computing step 43, in accordance with some embodiments of the present invention.

In some embodiments, measure-computing step 43 begins with a signal-averaging sub-step 44. At signal-averaging sub-step 44, the processor computes an average signal for each of the stimulated cortexes, by averaging the received signals over the electrodes and stimulations. Typically, the processor first computes an average for each electrode by averaging over the stimulations, and then averages these per-electrode averages.

Next, the processor, at a measure-computing sub-step 46, computes one or more measures of neurophysiological activity, such as an early waveform adherence measure and/or a cortical excitability measure, from each of the average signals. Finally, at a measure-averaging sub-step 48, the processor computes an average of each of these measures.

For example, assuming the right and left primary motor cortex and the right and left dorsolateral prefrontal cortex were stimulated, the processor may compute four average signals at signal-averaging sub-step 44. Next, at measure-computing sub-step 46, the processor may compute an early waveform adherence measure and a cortical excitability measure for each of these average signals. Finally, at measure-averaging sub-step 48, the processor may compute an average of the four early waveform adherence measures and another average of the four cortical excitability measures.

Alternatively to averaging each of the measures at measure-averaging sub-step 48, the processor computes a weighted average, a maximum, a minimum, or any other statistic for each of the measures.

Alternatively, the processor averages the signals over all of the cortexes, and then computes the measure for the single average signal. As yet another alternative, the processor computes the measure for each of the signals, and then computes the average (or another statistic) of the measures.

### Experimental Results

Reference is now made to FIGS. 4A-4H, which show experimental results demonstrating the utility of embodiments of the present invention. Each of FIGS. 4A-4H shows the mean values of a measure of neurophysiological activity obtained for multiple cohorts of subjects following stimulation of the left primary motor cortex, with error bars 54 indicating the associated standard deviations.

FIGS. 4A-4B show that the cortical excitability and early waveform adherence (WFA) were lower, by a statistically-significant margin, for a cohort of 166 subjects having cerebral small vessel disease, as determined from MRI images, relative to a cohort of 136 normal subjects. Thus, FIGS. 4A-4B demonstrate that cerebrovascular disease (e.g., cerebral small vessel disease) can be diagnosed based on cortical excitability and/or early waveform adherence, e.g., by comparing one of these measures to a threshold or both of these measures to respective thresholds.

FIGS. 4C-4D show the cortical excitability and early waveform adherence for three cohorts of subjects having different respective severities of leukoaraiosis, as determined from MRI images (in which leukoaraiosis is manifested as white matter hyperintensity): 173 subjects having grade 0 (G0), 225 subjects having grade 1 (G1), and 86 subjects having grade 2 or higher (G2+). (The grades are per the Fazekas scale, with grade 0 corresponding to no leukoaraiosis.) As shown, both measures decreased, by a statistically-significant margin, with increasing severity.

FIGS. 4E-4F show that the cortical excitability and early waveform adherence were lower, by a statistically-significant margin, for a cohort of 53 subjects having lacunar infarction, as determined from MRI images, relative to a cohort of 431 subjects without lacunar infarction. Similarly, FIGS. 4G-4H show that the cortical excitability and early waveform adherence were lower for a cohort of 77 subjects having cerebral microbleeds, as determined from MRI images, relative to a cohort of 407 subjects without microbleeds, with the difference being of greater statistical significance for cortical excitability.

Thus, FIGS. 4C-4H demonstrate that an indication for cerebrovascular disease can be identified based on cortical excitability and/or early waveform adherence, e.g., by comparing one of these measures to a threshold or both of these measures to respective thresholds. FIGS. 4C-4D additionally demonstrate that the severity of the cerebrovascular disease (e.g., the severity of leukoaraiosis) can be determined based on cortical excitability and/or early waveform adherence, e.g., by comparing one of these measures to a threshold or both of these measures to respective thresholds.

Reference is now made to FIGS. 5A-5C, which show additional experimental results demonstrating the utility of embodiments of the present invention. Each of FIGS. 5A-5C shows the mean values of cortical excitability obtained, following stimulation of the left primary motor cortex, for multiple cohorts of subjects having different respective medial temporal atrophy severities, with error bars 54 indicating the associated standard deviations. Each medial temporal atrophy severity is indicated by a score (per Schelten's scale) on the horizontal axis, with “MTA 0” corresponding to no medial temporal atrophy and a greater MTA score corresponding to a greater severity of medial temporal atrophy. (Each value is represented by a point in FIGS. 5A-5C, rather than a bar as in FIGS. 4A-4H.)

The results in FIG. 5A were obtained for a mix of cognitively-normal and cognitively-impaired subjects. There were 278 subjects with MTA 0, 131 with MTA 1, and 32 with MTA≥2. FIG. 5A shows that the cortical excitability decreases, in a statistically-significant manner, as the severity increases. FIG. 5A thus demonstrates that medial temporal atrophy, and optionally the severity thereof, can be diagnosed based on cortical excitability.

FIG. 5B shows the relationship between cortical excitability and medial temporal atrophy severity for cognitively-normal subjects, with the sizes of the cohorts being 225 subjects for MTA 0, 88 subjects for MTA 1, and 19 subjects for MTA≥1.5. FIG. 5C shows this relationship for cognitively-impaired subjects, with the sizes of the cohorts being 30 subjects for MTA 0, 27 subjects for MTA 1, and 10 subjects for MTA≥1.5. (In FIG. 5C, the mean cortical excitability for MTA≥1.5 was not statistically significant, due to the small size of the cohort.) As can be seen from FIGS. 5B-5C, cortical excitability decreases with cognitive impairment; for example, in FIG. 5B, the mean cortical excitability for MTA 0 is approximately 4, whereas in FIG. 5C, the mean cortical excitability for MTA 0 is approximately 3.6. FIGS. 5B-5C thus demonstrate that medial temporal atrophy, and optionally the severity thereof, can be diagnosed based on cortical excitability and, optionally, an input indicating whether the patient is cognitively impaired.

Embodiments of the invention described herein can take the form of a computer program product accessible from a computer-usable or computer-readable medium (e.g., a non-transitory computer-readable medium) providing program code for use by or in connection with a computer or any instruction execution system, such as computer processor 32. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 32) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 32) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 32 is typically a hardware device programmed with computer program instructions to produce a special purpose computer. For example, when programmed to perform the algorithms described with reference to the figures, computer processor 32 typically acts as a special purpose diagnostics computer processor. Typically, the operations described herein that are performed by computer processor 32 transform the physical state of a memory, which is a real physical article, to have a different magnetic polarity, electrical charge, or the like depending on the technology of the memory that is used. For some embodiments, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system, comprising:
multiple electrodes configured to record respective signals produced, by a brain of a patient, in response to magnetic stimulation of the brain; and
a processor, configured to:
receive the signals,
by processing the signals, ascertain that the patient has cerebrovascular disease, and
output an output indicating that the patient has cerebrovascular disease.

2. The system according to claim 1, wherein the magnetic stimulation includes magnetic stimulation of a primary motor cortex of the brain.

3. The system according to claim 1, wherein the magnetic stimulation includes magnetic stimulation of a dorsolateral prefrontal cortex of the brain.

4. The system according to claim 1, wherein the processor is further configured to identify a severity of the cerebrovascular disease by processing the signals, and wherein the output indicates the severity.

5. The system according to claim 1, wherein the processor is configured to process the signals by:
computing one or more measures of neurophysiological activity exhibited in the signals, and
ascertaining that the patient has cerebrovascular disease based on the measures.

6. The system according to claim 5, wherein the measures of neurophysiological activity include a waveform adherence measure, which is based on a similarity between a waveform of a portion of the signals and a waveform of a corresponding portion of a benchmark signal.

7. The system according to claim 6, wherein the measures of neurophysiological activity include an early waveform adherence measure, which is based on a similarity between a waveform of a portion of the signals and a waveform of a corresponding portion of a benchmark signal, the portion of the signals comprising portions of signals that begins less than 50 ms from the starts of respective stimulation pulses of the brain, the portions of the signals having durations of between 120 and 160 ms.

8. The system according to claim 5, wherein the measures of neurophysiological activity include a cortical excitability measure, which is based on an amplitude of the signals.

9. The system according to claim 8, wherein the cortical excitability measure is determined by processing the signals to generate at least one measure of the signals selected from the group consisting of: an integral of the signals, a statistic of the amplitude of the signals, and a mean average deviation of the amplitude of the signals.

10. The system according to claim 1, wherein the processor is further configured to identify subset indications of multiple indications of cerebrovascular disease by processing the signals, and wherein the output indicates that the patient has at least one of the subset indications of the multiple indications.

11. The system according to claim 10, wherein the subset indications of the multiple indications include leukoaraiosis, cerebral microbleeds, and lacunar infarction.

12. A system, comprising:
multiple electrodes configured to record respective signals produced, by a brain of a patient, in response to magnetic stimulation of the brain; and
a processor, configured to:
receive the signals,
by processing the signals, ascertain that the patient has medial temporal atrophy, and
output an output indicating that the patient has medial temporal atrophy.

13. The system according to claim 12, wherein the magnetic stimulation includes magnetic stimulation of a primary motor cortex of the brain.

14. The system according to claim 12, wherein the magnetic stimulation includes magnetic stimulation of a dorsolateral prefrontal cortex of the brain.

15. The system according to claim 12, wherein the processor is configured to process the signals by:
computing one or more measures of neurophysiological activity exhibited in the signals, and
ascertaining that the patient has medial temporal atrophy based on the measures.

16. The system according to claim 15, wherein the measures of neurophysiological activity include a cortical excitability measure, which is based on an amplitude of a portion of the signals.

17. The system according to claim 16, wherein the cortical excitability measure is determined by processing the signals to generate at least one measure of the signals selected from the group consisting of: an integral of the signals, a statistic of the amplitude of the signals, and a mean average deviation of the amplitude of the signals.

18. The system according to claim 12, wherein the processor is further configured to identify a severity of the medial temporal atrophy by processing the signals, and wherein the output indicates the severity.

19. The system according to claim 18, wherein the processor is configured to identify the severity based on an input indicating whether the patient is cognitively impaired.

20. A method, comprising:
receiving multiple signals, which were produced by a brain of a patient in response to magnetic stimulation of the brain and were recorded by respective electrodes;
by processing the signals, ascertaining that the patient has medial temporal atrophy; and
outputting an output indicating that the patient has at least one condition selected from the group consisting of: medial temporal atrophy and cerebrovascular disease.
